(19) United States
(12) Patent Application Publication
(10) Pub. No.: US 2024/0131367 A1
(43) Pub. Date: Apr. 25, 2024

(54) ULTRASOUND TREATMENT SYSTEM FOR THE BRAIN

(71) Applicant: INTELLIGENZA TRASPARENTE S.R.L., Monza (IT)

(72) Inventor: Alessandro Carmelo ROTILIO, Milano (IT)

(21) Appl. No.: 18/546,632

(22) PCT Filed: Jul. 8, 2021
(86) PCT No.: PCT/IB2021/056183

§ 371 Date: Aug. 15, 2023

(30) Foreign Application Priority Data
Feb. 17, 2021 (IT) 102021000003695

(51) Int. Cl. A61N 7/00; A61B 8/00; A61B 8/08

(52) U.S. Cl. CPC A61N 7/00; A61B 8/0808; A61B 8/4209; A61B 8/481; A61B 8/54; A61N 2007/0026; A61N 2007/0039; A61N 2007/0052

(57) ABSTRACT

A system for ultrasound brain treatment through modulation and control of emission of an ultrasound beam. The ultrasound beam includes an ultrasound probe, an ultrasound device, and a processing unit. The ultrasound probe performs an ultrasound scan and collects a stream of ultrasound images of a part of a patient's brain. The ultrasound device selectively modulates and controls the emission of an ultrasound beam on the basis of a desired treatment procedure for the part of the brain. The processing unit, which is in data communication with the ultrasound probe and with the ultrasound device, includes a sampling module, a graphics processing module, and a calculation module. The sampling module samples the stream of ultrasound images collected by the ultrasound probe. The graphic processing module performs a first step of graphic processing of the sampled frames by generating an analysis grid composed of a plurality of cells and elaborating a distribution map of targets to be treated with ultrasound within the part of the patient's brain. The distribution map of targets refers to the plurality of cells. The calculation module determines the ultrasound modulation and control parameters of the ultrasound device on the basis of the desired treatment procedure and the distribution map of targets.

### TECHNICAL FIELD

The present invention relates to a system for neurological and neurosurgical therapies based on the use of ultrasound.

In particular, the present invention relates to a system for ultrasound treatment of the brain through the modulation and control of the emission of an ultrasound beam in a selective and targeted manner, also in the presence of a contrast liquid containing microbubbles or other ultrasound-reagent substances.

### PRIOR ART

It is known that the ultrasound can generate therapeutic effects on the brain, such as, for example, the ablation of tissues or opening of the blood-brain barrier.

It is known that some of these effects can be generated with the use of an ultrasound contrast medium, by exploiting the agitation of the microbubbles making up the contrast medium or the cavitation thereof, both phenomena characterised by the release of energy inside the brain. However, it is known that an excessive release of energy may entail risks, for example of generating haemorrhages or non-reversible opening of the blood-brain barrier.

This element is a limitation to the use of ultrasound.

It is known that the devices that achieve the opening of the blood-brain barrier through the use of ultrasound, whether associated or not with microbubbles, are not capable of delivering ultrasound with parameters such as to render homogeneous the deposit of energy in the tissues to be treated.

They are in fact typically characterised by an indiscriminate emission of ultrasound.

This element can generate non-homogeneous openings of the blood-brain barrier, with areas of greater opening (safety risk) or areas of less opening (risk of poor effectiveness of the treatment).

There are known implantable devices, configured to deliver an ultrasound treatment on the brain in a precise manner.

However, implantable or transcranial devices do not allow the possibility of performing ultrasound scans.

### OBJECT OF THE INVENTION

The technical task at the basis of the present invention is to propose a system for ultrasound treatment of the brain through the modulation and control of the emission of an ultrasound beam which overcomes the above-mentioned drawbacks of the prior art.

In particular, the object of the present invention is to deliver ultrasound-based therapies in a “navigated” manner, i.e. guided by an ultrasound scan.

Another object of the present invention is to provide a system for ultrasound treatment of the brain through the modulation and control of the emission of an ultrasound beam that takes account of the density of the contrast liquid or of other ultrasound-reagent substances in the areas of the brain subjected to ultrasound treatment.

Another object of the present invention is to provide a system for ultrasound treatment of the brain through the modulation and control of the emission of an ultrasound beam that makes it possible to perform a selective treatment on areas subjected to treatment.

A further object of the present invention is to provide a system for ultrasound treatment of the brain through the modulation and control of the emission of an ultrasound beam that allows the cavitation to be rendered homogeneous in areas characterised by non-homogeneous anatomy. Another object of the present invention is to provide a system for ultrasound treatment of the brain through the modulation and control of the emission of an ultrasound beam that allows the agitation of fluids to be rendered homogeneous within areas characterised by different concentrations of such fluids or of the contrast media used.

A further object of the present invention is to provide a system for ultrasound treatment of the brain through the modulation and control of the emission of an ultrasound beam that allows the cavitation or the agitation of fluids or of the contrast medium to be rendered non-homogeneous where this is preferable for the purposes of the particular clinical treatment.

Another object of the present invention is to provide a system for ultrasound treatment of the brain through the modulation and control of the emission of an ultrasound beam that is an efficient, real-time system.

The stated technical task and the specified objects are substantially achieved by a system for ultrasound treatment of the brain through the modulation and control of the emission of an ultrasound beam comprising:
an ultrasound probe configured to perform an ultrasound scan and collect a stream of ultrasound images on a part to be treated of a patient's brain;
an ultrasound device configured to selectively modulate and control the emission of an ultrasound beam on the basis of a desired treatment procedure for the part of the brain;
a processing unit in data communication with said ultrasound probe and with said ultrasound device, comprising:
   a sampling module configured to sample the stream of ultrasound images collected by said ultrasound probe;
   a graphic processing module configured to perform a first step of graphic processing of said sampled frames by proceeding to:
      generate an analysis grid composed of a plurality of cells;
      elaborate a distribution map of targets to be treated with ultrasound within said part, said distribution map of targets referring to said plurality of cells;
   a calculation module configured to determine the ultrasound modulation and control parameters of said device on the basis of the desired treatment procedure and the distribution map of targets.

### BRIEF DESCRIPTION OF THE DRAWINGS

Additional features and advantages of the present invention will become more apparent from the approximate and thus non-limiting description of a preferred but not exclusive embodiment of a system for ultrasound treatment of the brain through the modulation and control of the emission of an ultrasound beam, as illustrated in the appended drawings, in which:

FIG. 1 schematically illustrates a block diagram of a system for ultrasound treatment of the brain through the modulation and control of the emission of an ultrasound beam in accordance with the present invention;

FIG. 2 illustrates an analysis grid generated by the system in FIG. 1;

FIG. 3 illustrates a positioning element adaptable to a skull portion to be treated, employable in the system in FIG. 1;

FIG. 4 illustrates the positioning element adaptable to a skull portion to be treated as per FIG. 3, wherein an ultrasound probe is also used;

FIG. 5 illustrates an ultrasound device that may be used in the system in FIG. 1, composed of ultrasound emitters arranged circularly, housed at the centre of which there is an ultrasound-transparent material to allow the passage of ultrasound of an ultrasound probe, coupled with an ultrasound-transparent material;

FIG. 6 illustrates a detail of the ultrasound device in FIG. 5, without the ultrasound-transparent material.

### DETAILED DESCRIPTION OF PREFERRED EMBODIMENTS OF THE INVENTION

In a first aspect, the present invention describes a system 1 for ultrasound treatment of the brain through the modulation and control of the emission of an ultrasound beam US. The system 1 comprises an ultrasound probe 10, an ultrasound device 20 and a processing unit 30 in data communication with said ultrasound probe 10 and with said ultrasound device 20. In one embodiment of the present invention, the ultrasound probe 10 and the ultrasound device 20, configured for the treatment of a patient's brain, are a single device.

The ultrasound probe 10 is configured to perform an ultrasound scan EC and collect a stream of ultrasound images D_IMM on a part A1 to be treated of a patient's brain.

The ultrasound device 20 is configured to selectively modulate and control the emission of an ultrasound beam US on the basis of a desired treatment procedure TR for the part A1 of a patient's brain.

In accordance with one aspect of the invention, the processing unit 30 is in data communication with the ultrasound probe 10 and with the ultrasound device 20 and comprises at least one sampling module 31, configured to sample the stream of ultrasound images D_IMM collected by said ultrasound probe 10, and a graphic processing module 32, configured to perform a first step F1 of graphic processing of said sampled frames D_CIM.

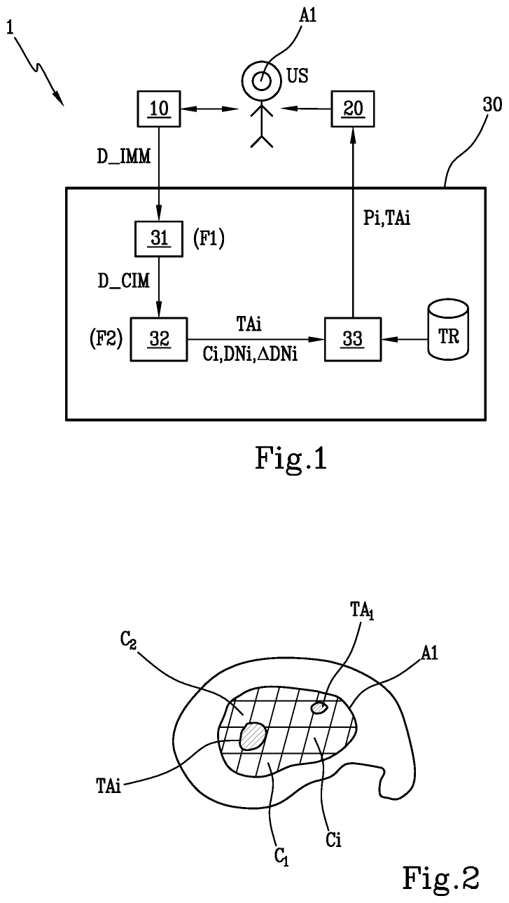

The graphic processing module 32 is configured to generate an analysis grid RA composed of a plurality of cells Ci, as illustrated in FIG. 2, and to elaborate a distribution map of targets TAi to be treated with ultrasound within said part A1. In particular, the distribution map of targets TAi is elaborated by the graphic processing module 32 on the basis of a spatial reference system of said plurality of cells Ci.

Finally, the processing unit 30 comprises a calculation module 33 configured to determine the ultrasound US modulation and control parameters Pi of said device 20 on the basis of the desired treatment procedure TR and the distribution map of targets TAi.

In general it should be noted that, in the present context and in the subsequent claims, the processing unit 30 will be understood as being divided into distinct functional modules (memory modules or operating modules) for the sole purpose of describing the functions thereof in a clear and complete manner.

The processing unit can consist of a single electronic device, suitably programmed to perform the functions described, and the different modules can correspond to hardware entities and/or routine software forming part of the programmed device.

Alternatively, or in addition, said functions can be carried out by a plurality of electronic devices over which the aforesaid functional modules can be distributed.

The processing unit 30 can further rely on one or more processors to execute the instructions contained in the memory modules.

If the desired treatment procedure TR for the patient's brain requires the introduction of a contrast medium, containing microbubbles, in the area of distribution of the targets TAi, the graphic processing module 32 is further configured to:
assign a density value DNi of the contrast medium in each cell Ci or in the cells Ci containing the targets TAi;
assign a value to the variation in density ΔDNi of the contrast medium;
elaborate a distribution map of the contrast medium within the analysis grid RA and/or of the areas of the targets TAi identified; and/or
elaborate a map of variation in the concentration of the contrast medium within the analysis grid RA.

In this manner it will be possible to control the ultrasound device 20 in such a way as to emit an ultrasound beam in the area of the brain to be selectively treated on the basis of the density values DNi and/or of the variation in density ΔDNi of the contrast medium.

In particular, the calculation module 33 is configured to generate the ultrasound US modulation and control parameters Pi of said ultrasound device 20 also taking account of the density value (DNi) of the contrast medium and/or of the value of the variation in density ΔDNi of the contrast medium in each cell Ci or in the cells Ci containing the targets TAi.

By way of non-limiting example, the calculation module 33 is configured to modulate and control the emission of an ultrasound beam US towards the targets TAi according to one or more of the following modes:
a. with the aim of rendering the cavitation of the microbubbles of the contrast medium homogeneous in areas characterised by a non-homogeneous anatomy;
b. with the aim of rendering the agitation of biological fluids homogeneous within areas characterised by different concentrations of said biological fluids or of the contrast medium;
c. with the aim of rendering the cavitation or agitation of biological fluids or contrast medium non-homogeneous where this is preferable for the purposes of the clinical treatment;
d. to enable the operator to select areas that will be subjected to treatment and areas that will not be subjected to treatment.

Advantageously, the ultrasound device 20 comprises one or more ultrasound emitters 21, each of which can be modulated and controlled individually and independently, and a control unit configured to receive said parameters Pi and the map of identified targets TAi from said calculation module 33 so as to be able to activate and modulate the various emitters 21 in different times and modes.

In one embodiment of the invention, the ultrasound device 20 is made in such a way as to produce both ultrasound images D_IMM of the patient's brain and therapeutic ultrasound.

As illustrated in the example in FIG. 4, the ultrasound device 20 is made in such a way as to enable the simultaneous use thereof with an ultrasound probe 10. In particular, in this example, the ultrasound device 20 is made in the shape of a ring or a rectangle, at whose centre the ultrasound probe 10 may be housed, and comprises, at the perimeter thereof, a plurality of ultrasound emitters 21, each of which can be modulated and controlled independently.

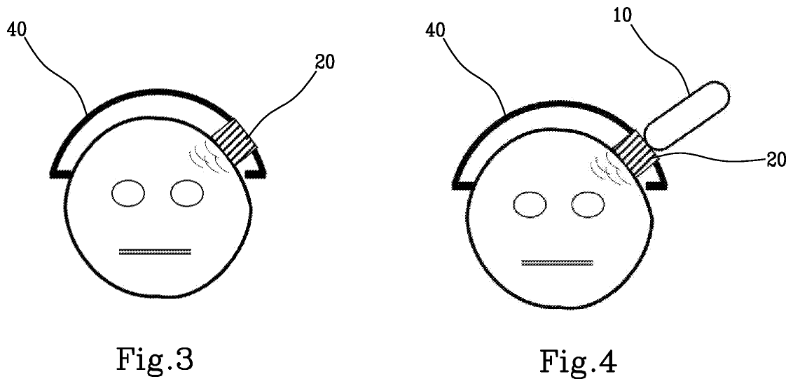

The system 1 of the invention can advantageously comprise a frame or positioning element 40 configured to removably maintain the ultrasound device 20 in position on the patient's skull in the area to be treated A1 and to simultaneously enable the ultrasound probe 10 to be positioned in such a way as to permit an ultrasound treatment guided by the ultrasound images D_IMM generated by the probe 10. The positioning element 40 represented in FIGS. 3 and 4 has the shape of a rigid or flexible semicircle, with a thickness suitable for keeping the ultrasound device 20 in position.

In this manner, thanks to the positioning element 40 it is possible to obtain a coincident reference system Rif for the treatment device 20 and the ultrasound probe 10.

The positioning element 40 is configured to removably receive said ultrasound probe 10 and/or said ultrasound device 20 and is likewise easily removable. In particular, the positioning element 40 is configured to maintain the ultrasound device 20 in an operating position between itself and a portion of the patient's skull to be treated with ultrasound US.

In order to enable the joint use of the ultrasound device 20 with the ultrasound probe 10, the positioning element 40 comprises at least one portion made with a deformable or semirigid ultrasound-transparent material so as not to interfere with the ultrasound probe 10.

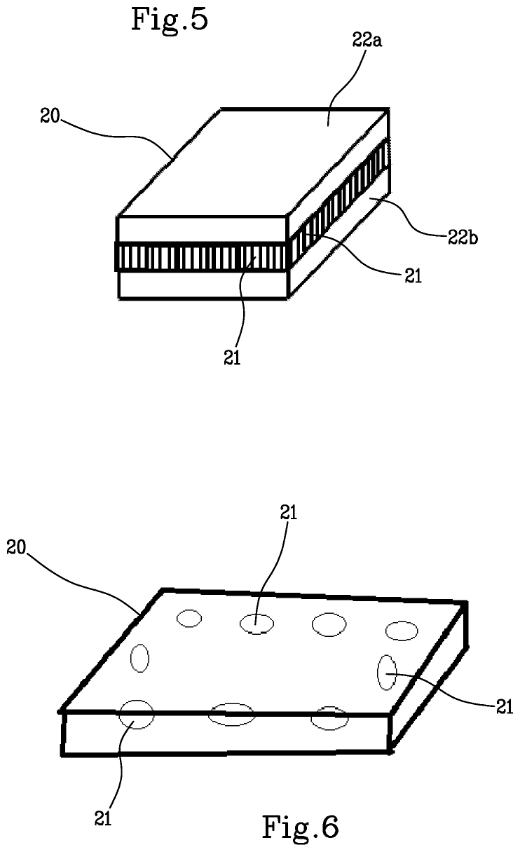

As illustrated in FIG. 5, the ultrasound device 20 comprises an ultrasound-transparent material 22b, positioned on at least one surface substantially parallel to a portion of the patient's skull. Preferably, a layer of said ultrasound-transparent material 22a also covers the surface of the ultrasound device 20 opposite the one resting on the patient's skull. By way of non-limiting example, that material is a gel.

In a second aspect, the present invention describes a method for ultrasound treatment of a patient's brain through the modulation and control of the emission of an ultrasound beam US, comprising the steps of:
performing, by means of an ultrasound probe 10, an ultrasound scan EC and collecting a stream of ultrasound images D_IMM on a part A1 to be treated of a patient's brain;
performing a sampling 31 of the stream of ultrasound images D_IMM collected by said ultrasound probe 10;
performing a first step F1 of graphic processing of said sampled frames D_CIM by proceeding to:
   generate an analysis grid RA composed of a plurality of cells Ci;
   elaborate a distribution map of targets TAi to be treated with ultrasound within said part A1, said distribution map of targets TAi referring to said plurality of cells Ci;
   determine the ultrasound US modulation and control parameters Pi of said device 20 on the basis of the desired treatment procedure TR and the distribution map of targets TAi elaborated in the preceding step;
selectively modulating and controlling the emission of an ultrasound beam US on the basis of the desired treatment procedure TR for said part A1 to be treated, using the ultrasound modulation and control parameters Pi.

In particular, if the ultrasound device 20 is composed of a plurality of emitters 21, it will be possible to modulate and control the ultrasound beam emitted by each emitter on the basis of the target and the treatment TR to be performed.

If the desired treatment procedure TR for the patient's brain requires the introduction of a contrast medium, containing microbubbles, in the area of distribution of the targets TAi, the method according to the invention further comprises the steps of:
assigning a density value DNi of the contrast medium in each cell Ci or in the cells Ci containing the targets TAi;
   assigning a value to the variation in density ΔDNi of the contrast medium;
   elaborating a distribution map of the contrast medium within the analysis grid RA and/or of the areas of the targets TAi identified; and/or
   elaborating a map of variation in the concentration of the contrast medium within the analysis grid RA.

In such a case, the step of determining the ultrasound US modulation and control parameters Pi of said ultrasound device 20 also takes account of the presence of the contrast medium, i.e. of one or more of the steps described in the previous paragraph.

In a third aspect, the present invention relates to a computer program which implements one or more steps of the method.

As the skilled person can easily understand, the invention makes it possible to overcome the drawbacks highlighted previously with reference to the prior art.

In particular, the present invention enables ultrasound-based therapies to be delivered in a “navigated” manner, i.e. guided by an ultrasound scan. Furthermore, the present invention makes it possible to perform a selective treatment of areas of a patient's brain subjected to treatment.

It is clear that the specific characteristics are described in relation to different embodiments of the invention by way of non-limiting example. A person skilled in the art will obviously be able to introduce further modifications and variants to the present invention in order to satisfy specific contingent needs. For example, the technical features described in relation to one embodiment of the invention can be extrapolated therefrom and applied to other embodiments of the invention. Moreover, such modifications and variants are contained in the scope of protection of the invention, as defined by the following claims.

1-11. (canceled)

12. A system for ultrasound treatment of a patient's brain comprising:
an ultrasound probe configured to perform an ultrasound scan and collect a stream of ultrasound images on a portion of the patient's brain;
an ultrasound device configured to selectively modulate and control an emission of an ultrasound beam based on a desired treatment procedure for the portion of the patient's brain; and
a processing unit in data communication with said ultrasound probe and said ultrasound device, the processing unit comprising:
(a) a sampling module configured to sample the stream of ultrasound images collected by said ultrasound probe;
(b) a graphic processing module configured to perform graphic processing of sampled frames by:
(i) generating an analysis grid composed of a plurality of cells; and
(ii) elaborating a distribution map of targets to be treated with ultrasound within said portion of the patient's brain, said distribution map of targets referring to said plurality of cells; and
(c) a calculation module configured to determine ultrasound modulation and control parameters of said ultrasound device based on the desired treatment procedure and the distribution map of targets.

13. The system according to claim 12, wherein the desired treatment procedure for the patient's brain comprises an introduction of a contrast medium containing microbubbles in an area of the distribution map of the targets and said graphic processing module is further configured to:
assign a density value of the contrast medium in cells containing the targets;
assign a value to the variation in density of the contrast medium;
elaborate a distribution map of the contrast medium within the analysis grid and of the areas of the targets; and
elaborate a map of variation in a concentration of the contrast medium within the analysis grid.

14. The system according to claim 13, wherein said calculation module is further configured to determine said ultrasound modulation and said control parameters of said ultrasound device by the density value of the contrast medium and the value of the variation in density of the contrast medium in the cells containing the targets.

15. The system according to claim 13, wherein said calculation module is configured to determine the ultrasound modulation and control parameters of said ultrasound device according to at least one of the following modes:
a. for a homogeneous cavitation of the microbubbles of the contrast medium in areas characterised by a non-homogeneous anatomy;
b. for a homogeneous agitation of biological fluids within areas characterised by different concentrations of such biological fluids or of the contrast medium;
c. for an inhomogeneous cavitation or agitation of biological fluids or contrast media where preferable for clinical treatment purposes; and
d. enabling the operator to select areas that will be subjected to treatment and areas that will not be subjected to treatment.

16. The system according to claim 12, further comprising a positioning element removable from a patient's skull portion, configured to receive said ultrasound probe or said ultrasound device to provide a coincident reference system.

17. The system according to claim 16, wherein the positioning element comprises at least one portion made with a deformable or semi-rigid ultrasound-transparent material and is configured to maintain the ultrasound device in an operating position between the positioning element and the patient's skull portion.

18. The system according to claim 12, wherein the ultrasound device comprises:
one or more ultrasound emitters, each of which can be modulated and controlled individually and independently, and
a control unit configured to:
receive said control parameters and said distribution map of targets from said calculation module to activate and modulate the one or more ultrasound emitters in different times and modes.

19. The system according to claim 12, wherein the ultrasound device can produce ultrasound images of the patient's brain and of a therapeutic ultrasound.

20. The system according to claim 12, wherein the ultrasound device enables simultaneous use of the ultrasound device with the ultrasound probe.

21. The system according to claim 20, wherein the ultrasound device is shaped as a ring, at whose center the ultrasound probe is housed, and is made with ultrasound-conductive material so as not to interfere with the ultrasound probe.

22. The system according to claim 12, wherein the ultrasound device comprises an ultrasound-transparent material on at least one surface to be placed parallel to a portion of the patient's skull.
